(19) United States
(12) Patent Application Publication
(10) Pub. No.: US 2011/0091375 A1
(43) Pub. Date: Apr. 21, 2011

(54) USE OF THE COMBINATION COMPRISING TEMOZOLOMIDE AND TNF-ALPHA FOR TREATING GLIOBLASTOMA

(75) Inventors: Ralph R. Weichselbaum, Chicago, IL (US); Donald W. Kufe, Wellesley, MA (US); Bakhtiar Yamini, Chicago, IL (US)

(73) Assignees: THE UNIVERSITY OF CHICAGO, Chicago, IL (US); DANA-FARBER CANCER INSTITUTE, INC., Boston, MA (US)

(21) Appl. No.: 12/971,953

(22) Filed: Dec. 17, 2010

Related U.S. Application Data
(63) Continuation of application No. 11/574,108, filed on May 11, 2007, now abandoned.
(60) Provisional application No. 60/604,251, filed on Aug. 25, 2004.

(51) Int. Cl. A61K 51/00; C12N 5/09; A61K 38/19; A61K 31/4188; A61K 35/76; A61P 35/00

(52) U.S. Cl. 424/1.11; 435/375; 424/85.1; 514/393; 424/93.2

(57) ABSTRACT

Disclosed are methods of synergistically inhibiting growth of a glioma cell comprising contacting the cell with temozolomide and TNFα, or with temozolomide, TNFα, and radiation. Also disclosed are methods of synergistically inhibiting growth of a glioma in a human cancer patient comprising administering temozolomide and TNFα, or with temozolomide, TNFα, and radiation. Pharmaceutical combinations and therapeutic combinations suitable for use in the methods of the invention are also disclosed.

### CROSS-REFERENCE TO RELATED APPLICATIONS

This application claims priority to U.S. Provisional Application No. 60/604,251, filed Aug. 25, 2004.

### STATEMENT REGARDING GOVERNMENT-SPONSORED RESEARCH

This invention was made with U.S. Government support under grant ______ awarded by ______. The Government has certain rights in the invention.

### INTRODUCTION

Despite aggressive treatment of malignant glioma, there has been little improvement over the past 30 years in the survival of patients with malignant gliomas. Radiation therapy (IR) remains the mainstay of post-surgical management. Recently, the concurrent use of the oral alkylating agent temozolomide (TMZ) with IR has been shown to modestly increase prognosis in patients who have undergone complete surgical resection (Stupp, R. et al. (2005) N Engl J Med 352:987-996). Promising investigational targeted therapies (Castro, M. G. et al. (2003) Pharmacol Ther 98:71-108), such as targeted toxins, monoclonal antibodies or immune mediated approaches, have yet to make a significant clinical impact. A number of factors account for the poor response of malignant brain tumors to therapy, including the intrinsic resistance of glioma cells to DNA damage-induced cytotoxicity (Taghian, A. et al. (1995) Int J Radiat Oncol Biol Phys 32:99-104) (Johnstone, R. W. et al. (2002) Cell 108:153-164) and the normal tissue toxicity produced by currently employed therapeutic agents. Investigation of combination treatment strategies that activate complementary cytotoxic pathways is an important aspect of developing anti-cancer treatments that overcome resistance to treatment and improve patient prognosis (Vivo, C. et al. (2003) J Biol Chem 278:25461-25467).

TMZ is a monofunctional alkylating agent with a favorable toxicity profile commonly used in the treatment of malignant glioma. Although the combined use of TMZ and IR is now a preferred regimen for the treatment of both newly diagnosed and recurrent glioblastoma, the prognosis for people with malignant glioma remains dismal.

Therefore, there exists a need in the art for improved methods, pharmaceutical, and therapeutic combinations for treating people with malignant glioma.

### SUMMARY OF THE INVENTION

In one aspect, the present invention provides a method of synergistically inhibiting growth of a glioma cell comprising contacting the cell with temozolomide and TNFα.

In another aspect, the invention provides a method of synergistically inhibiting growth of a glioma in a human cancer patient comprising administering to the patient temozolomide and a vehicle comprising or expressing TNFα, wherein the vehicle is administered directly to the glioma.

In yet another aspect, the invention includes a method of synergistically inhibiting growth of a glioma in a human cancer patient comprising administering to the patient temozolomide, a vehicle comprising or expressing TNFα, and radiation, wherein the vehicle and irradiation are administered directly to the glioma.

Also provided are a pharmaceutical combination comprising temozolomide and a vehicle comprising or expressing TNFα, and a therapeutic combination comprising temozolomide, a vehicle comprising or expressing TNFα, and radiation.

### BRIEF DESCRIPTION OF THE DRAWINGS

FIG. 1 shows the amount of TNFα produced by U87 malignant glioma cells transfected with adenovirus expressing TNFα under the control of an Egr-1 promoter in vitro (FIG. 1A) and in vivo (FIG. 1B) in response to exposure to TMZ.

FIG. 2A shows the percent cell viability, as measured by the tryptan blue dye exclusion method, of U87 malignant glioma cells subjected to different treatments; FIG. 2B shows the optical densities (490 nm), obtained using the MTS colorimetric assay, for U87 malignant glioma cell populations exposed to different treatments.

FIG. 3A shows the fractional tumor volume (V/V₀) of hindlimb glioma tumors as a function of time post exposure to different treatments; FIG. 3B shows the percent survival (in days) for populations of mice with hindlimb glioma tumors exposed to different treatments as a function of time.

FIG. 4 shows apoptosis, as measured by TUNEL positive U87 glioma cells/10⁻⁶ mm², as a function of treatment.

FIG. 5 shows Kaplan-Meier survival curves of nude mouse intracranial xenografts.

### DETAILED DESCRIPTION OF THE INVENTION

The present invention provides methods for synergistically inhibiting or reducing the growth of malignant glioma cells using pharmaceutical or therapeutic combinations. The method includes use of a pharmaceutical combination of temozolomide (TMZ) and tumor necrosis factor-alpha (TNFα), or a therapeutic combination comprising TMZ, TNFα, and radiation therapy (IR). Thus, the present invention provides a therapeutic approach to treating malignant glioblastoma.

Before any embodiments of the invention are explained in detail, it is to be understood that the invention is not limited to the details of the invention set forth in the following description or illustrated in the appended figures. The invention is capable of other embodiments and of being practiced or of being carried out in various ways. Also, it is to be understood that the phraseology and terminology used herein is for the purpose of description and should not be regarded as limiting.

As used herein, the term “synergistically inhibits” means that the total inhibitory effect of the agents administered is greater than the sum of the individual inhibitory effects of the agents.

The term “contacting” is used herein interchangeably with the following: combined with, treating, added to, mixed with, passed over, incubated with, etc.

As used herein, “radiation” or “radiation therapy” refers to all known and appropriate forms of radiant energy (e.g., alpha, beta, gamma and x-rays as well as protons) that are commonly used in cancer treatment and delivered by any known method of delivery, for example, from an external source (beam), radiation from a radiation source implanted proximal to the tumor, radiation from a radionuclide attached to monoclonal antibodies or a compound that targets the cancer, radiation in a gamma knife, 3D conformal radiation, and radiation in steriotactic radiosurgery.

The therapeutic or pharmaceutical combinations of the present invention are meant to refer to a combination therapy or treatment by any administration route in which two or more therapeutic agents, including modalities such are radiation, are administered to cells, to a patient or to a subject. For combination treatment with more than one active agent, where the active agents are in separate formulations or modalities, the active agents can be administered concurrently, or they each can be administered at separately staggered times. The agents may be administered simultaneously or sequentially, as long as they are given in a manner sufficient to allow both agents to achieve effective concentrations in the cell or body. The agents may be administered by different routes, e.g., one agent may be administered intravenously while a second agent is administered intramuscularly, intravenously or orally.

In time-sequential administration, one agent may directly follow administration of the other or the agents may be give episodically, i.e., one can be given at one time followed by the other at a later time, e.g., within 2-3 days, or one can be given daily while another is given episodically, e.g., every 2-3 days. Suitable time-sequential administration in accordance with the present invention is detailed in the Examples below.

The pharmaceutical compositions used in the pharmaceutical or therapeutic combinations of this invention may be administered orally, parenterally, by intratumoral injection, by inhalation spray, topically, rectally, nasally, buccally, vaginally or via an implanted reservoir. Oral administration or administration by injection is most common. The pharmaceutical compositions of this invention may contain any conventional non-toxic pharmaceutically-acceptable carriers, adjuvants or vehicles.

The dosage amount of the compositions in accordance with the present invention for treating a patient is an amount sufficient to inhibit or reduce growth of a glioma cell or tumor. Specific dosage and treatment regimens for any particular patient will depend upon a variety of factors, including the activity of the specific compound employed, the age, body weight, general health status, sex, diet, time of administration, rate of excretion, drug combination, the severity and course of the infection, the patient's disposition to the infection and the judgment of the treating physician. Thus, the number of variables in regard to an individual treatment regimen is large, and a considerable range of doses is expected.

In an illustrated embodiment, the invention provides a method of synergistically inhibiting or reducing the growth of glioma cells by contacting the cells with a combination TMZ and TNFα, i.e., the cells are treated or contacted with both agents. Temozolomide is an imidazotetrazine derivative having the structure:

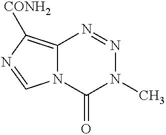

Temozolomide is commonly and conveniently administered orally in capsule form. However, it should be appreciated that TMZ could be also be administered by any other suitable means, e.g., intraperitoneally (IP), as shown in the Examples below.

In the methods of the invention, glioma cells are contacted with TNFα by contacting the glioma cells with a vehicle comprising or expressing TNFα. Such vehicles may include, for example, a liposome or nanoparticle comprising the TNFα polypeptide, or an expression vector, such as a viral vector comprising a polynucleotide encoding the TNFα polypeptide operably linked to a promoter functional in the host cell. Preferably, the promoter is an inducible promoter responsive to TMZ and/or IR, such as a promoter comprising the CArG elements of the Egr-1 promoter. Suitably, the viral vector is an adenovirus vector, preferably a replication defective adenovirus vector.

In the Examples, Ad.Egr-TNF was injected into the glioma, followed by intraperitoneal administration of TMZ, or IR and TMZ. As one of ordinary skill in the art will appreciate, the order in which various components of the pharmaceutical combination or therapeutic combination are administered is not critical. Some routine optimization may be involved to insure that TMZ and/or IR are present at a level sufficient to induce expression of TNFα, or that TMZ is present at a time and concentration effective to inhibit NFκB, or to act synergistically with TNFα to increase apoptosis or cytotoxicity.

TNFα is a prototypical death ligand and induces cytotoxicity via the extrinsic apoptosis cascade following binding to its cell surface receptor. However, TNFα-induced cytotoxicity is abrogated by preferential activation of the pro-survival transcription factor, nuclear factor-κB (NF-κB) (Karin, M. and Lin, A. (2002) Nat Immunol 3:221-227), which confers resistance to the cells. Activation of NF-κB has also been shown to mediate resistance to other genotoxic stressors such as IR (Wang, C. Y. et al. (1996) Science 274:784-787), and inhibition of TNFα- or IR-induced NF-κB has been shown to potentiate the cytotoxicity of these agents (Beg, A. A. and Baltimore, D. (1996) Science 274-782-784) (Van Antwerp, D. J. et al. (1996) Science 274:787-789) (Yamagishi, N. et al. (1997) Int J Radiat Biol 72:157-162) (Miyakoshi, J. and Yagi, K. (2000) Br J Cancer 82:28-33).

Briefly, the NF-κB family consists of five structurally related proteins the most abundant form of which consists of the heterodimer of p50 (NF-κB1) and p65 (RelA). In unstimulated cells, NF-κB is sequestered in the cytosol bound to inhibitor of-κB protein (IκBα). Following TNFα stimulation, IκBα is phosphorylated and degraded releasing the NF-κB subunits which translocate into the nucleus, bind to DNA and activate transcription. Phosphorylation of IκBα occurs following activation of the IκBα kinase (IκK) complex. In addition to IκBα degradation, NF-κB transcriptional activity has also been shown to be regulated by post-translational modification of the p65 subunit.

As described in the Examples below, the effects of combinations of TMZ and Ad.Egr-TNF, and TMZ, IR and Ad.Egr-TNF were evaluated in mouse hindlimb and intracranial malignant glioma xenografts, two models of human malignant glioma. Therapy was shown to be significantly more effective than the current standard anti-glioma regimen of TMZ and IR, by several different criteria, including extended survival, reduced tumor volume, enhanced apoptosis, and enhanced cytotoxicity. TMZ-mediated inhibition of TNFα- and IR-induced NF-κB activation is responsible, at least in part, for the enhanced results obtained using these combinations. Furthermore, enhanced interaction between TNFα and TMZ leads to the accumulation of reactive oxygen species (ROS), resulting in delayed c-Jun N-terminal kinase (JNK) activation that mediates tumor cell apoptosis.

The following non-limiting Examples are intended to be purely illustrative.

### EXAMPLES

### Example 1

### Reagents and Cells

TMZ was supplied by Schering Corporation (Kenilworth, N.J., USA) and was dissolved in DMSO with the final concentration not exceeding 0.1% (v/v). DMSO and human TNFα were obtained from Sigma (St. Louis, Mo., USA). N-acetylcystein (NAC) was obtained from Roxane Laboratories, Inc. (Columbus, Ohio, USA). Annexin V-FITC apoptosis detection kit II was manufactured by BD Pharmingen (San Jose, Calif., USA). Hydroethidine (HE) was purchased from Molecular Probes, Invitrogen Detection Technologies (Eugene, Oreg., USA). SP600125 was purchased from (EMD Bioscience, San Diego, Calif., USA). Human glioblastoma cell lines: U87 MG, T98MG, U251, pancreatic cancer cells: Panel, MIAPaCa-2 and BxPC-3 and esophageal cancer cells: Seg-1 were purchased from American Type Culture Collection and cultured in DMEM supplemented with 10% FBS (Intergen Co., Purchase, N.Y., USA), penicillin (1001 U/mL), and streptomycin (100 μg/mL) (Invitrogen Life Technologies, Carlsbad, Calif., USA) at 37° C. and 5% CO₂.

### Example 2

### Plasmids and Virus

The expression vectors pRC-CMV-p65, pRC-CMV and green fluorescent protein (GFP) have been previously described (Tang, F. et al. (2002) Mol Cell Biol 22:8571-8579). The NF-κB luciferase reporter construct, Ig-κB-Luc, containing three repeats of the immunoglobulin κ-light chain enhancer KB site and the Egr-1 promoter luciferase construct, pE425 GL3, have also been previously described (Park, J. O. et al. (2002) J Clin Invest 110:403-410) (Kanno, T. et al. (1995) J Biol Chem 270:11745-11748). The replication incompetent adenoviral vector, Ad.Egr-TNF, was described in U.S. Provisional Application No. 60/604,251 (Yamini, B., et al. (2004) Cancer Res 64:6381-6384). Ad.Egr-TNF (GenVec Inc., Gaithersburg, Md.) was stored at −80° C. and diluted in formulation buffer to the appropriate concentration. Temozolomide (Schering Corporation, Kenilworth, N.J.) was dissolved in DMSO with the final concentration not exceeding 0.1% (v/v).

### Example 3

TNFα Induction In Vitro

10⁶ U87 cells were plated and incubated overnight. The cells were then infected with Ad.Egr-TNF at a multiplicity of infection (MOI) of 100 for 3 h at 37° C. After incubation, 3.8 mL of complete media with or without TMZ was added. Conditioned media were harvested at 48 h after treatment and human TNFα production was quantified using a Quantikine ELISA kit (R&D System Inc., Minneapolis, Minn.).

### Example 4

TNFα Induction In Vivo

U87 cells (5×10⁶) in 100 DMEM were injected subcutaneously (sc) into the right hindlimb of nude mice. When tumors reached an average size of 200 mm³ (length×width×thickness/2), the tumor-bearing mice were randomized into 4 groups: 1. Untreated Control (UTC); 2. Ad.Egr-TNF alone; 3. TMZ alone; 4. Ad.Egr-TNF and TMZ. Ad.Egr-TNF was injected intratumorally (IT) at a dose of 2×10⁸ particle units (pu) each day. Two doses of TMZ were given: 2.5 mg/kg/day and 5 mg/kg/day by intraperitoneal (IP) injection 3 h after vector. Four consecutive daily IT and IP injections were given, control animals received IT and IP serum free medium (SFM). Animals were euthanized on day 2 and 4 (i.e., 48 h and 96 h after treatment initiation), tumors harvested, snap-frozen in liquid nitrogen and homogenized in RIPA buffer (150 mM NaCl, 10 mM Tris at pH 7.5, 5 mM EDTA at pH 7.5, 100 mM PMSF, 1 μg/mL leupeptin, and 2 μg/mL aprotinin). Protein was isolated and concentration measured using Protein Assay reagent (Bio-Rad Laboratories, Hercules, Calif.). TNFα levels in the supernatants were measured as described above.

### Example 5

### U87 Cell Viability Studies

Trypan Blue dye exclusion method was employed. 10⁴ U87 cells were plated and incubated at 37° C. overnight. Subsequently, the cells were contacted with media containing TNFα (10 ng/mL) and/or TMZ (100 μM), incubated for 3 h, and washed. At 24 h, 48 h, and 72 h following exposure to agent, the cells were trypsinized and the viable cell number/well determined using a hemocytometer. Cell viability at 72 h was verified using the MTS colorimetric assay, per the manufacturer's protocol (Cell Titer 96 Aqueous, One Solution cell proliferation assay; Promega Corporation, Madison, Wis., USA). Optical density was read at 490 nm using an ELISA microplate reader after 1.5 h, at 37° C. All of the studies were performed in triplicate.

### Example 6

### Xenograft Studies

Hindlimb Studies: U87 hindlimb xenografts were established as described above in Example 4. In one study, mice were randomized into four groups as described in Example 4 and treatment initiated (day 0). Ad.Egr-TNF (2×10⁸ pu) was injected IT twice a week for 4 total injections, and 5 mg/kg TMZ was given IP 3 h after each vector injection for a total of 20 mg/kg. The dose of TMZ used was approximately 0.2 LD₁₀ and was chosen to have modest anti-tumor effect but to not be curative based on previous studies (Friedman, H. S. et al. (1995) Cancer Res 55(13):2853-2857) and data from our lab showing LD₅₀ for IP TMZ to be approximately 500 mg/kg. Tumor volume was measured every 2-3 days. Fractional tumor volume (V/V₀ where V₀=volume on day 0) was calculated and plotted.

In a second study, tumor-bearing mice were randomized into eight treatment groups: untreated control (UTC); intratumoral (IT) Ad.Egr-TNF alone; intraperitoneal (IP) TMZ alone; IR alone; Ad.Egr-TNF and TMZ; Ad.Egr-TNF and IR; TMZ and IR; and Ad.Egr-TNF, TMZ and IR. Ad.Egr-TNF was administered IT at a dose of 2×10⁸ pu/10 μL twice a week for 2 weeks, IP TMZ was given 3 h after vector at 5 mg/kg to a total of 20 mg/kg. Animals were placed in Lucite chambers and given 5 Gy IR to the tumor 1 h before TMZ (on days when both TMZ and IR were administered), every 2-3 days to a total of 30 Gy. For all controls, animals were injected IT or IP with serum free medium (SFM) and animals were also placed in chambers without IR. Xenografts were measured twice a week using calipers, tumor volume was calculated, and fractional tumor volumes (V/V₀ where V₀=volume on day 0) were plotted.

Intracranial Studies: In two separate experiments, 5×10⁵ U87 cells were inoculated into the right caudate nucleus on day 0 using a screw guide technique (Lal, S. et al. (2000) J Neurosurg 92:326-333). In the first experiment, mice were randomized into four groups as described above in Example 4. On day 5, a single intracranial (IC) injection of 5×10⁸ pu Ad.Egr-TNF in 5 μl volume was made directly into the tumor using the screw guide technique. TMZ (5 mg/kg) was given IP 3 h after IC vector inoculation. Three additional IP TMZ injections were administered on consecutive days for a total dose of 20 mg/kg. Control animals received SFM IT and IP.

In a second experiment, mice were randomized into eight groups (untreated control (UTC); intratumoral (IT) Ad.Egr-TNF alone; intraperitoneal (IP) TMZ alone; IR alone; Ad.Egr-TNF and TMZ; Ad.Egr-TNF and IR; TMZ and IR; and Ad.Egr-TNF, TMZ and IR). A single dose of 5×10⁵ U87 cells was inoculated into the right caudate nucleus of each mouse on day 0 using a screw guide technique (Lal, S. et al. (2000) J Neurosurg 92:326-333). On day 5, 5×10⁸ pu Ad.Egr-TNF in 5 μL volume was injected once via the screw guide directly into the tumor. Beginning two hours after vector injection, 5 Gy IR was delivered to the tumor area and repeated every 2-3 days to a total of 30 Gy. TMZ (5 mg/kg) was given IP 3 h after vector inoculation and like doses were given daily for the next two days for a total dose of 15 mg/kg. Control animals received SFM IT and IP and were also placed in Lucite chambers. Daily assessment of animal appearance was made. Mice were followed until death or sacrificed when moribund. Mouse brains were harvested following intracardiac perfusion and fixed with 10% neutral buffered formalin. For TUNEL evaluation (see below) animals were sacrificed on day 7 following treatment (n=3 per group).

### Example 7

### Flow Cytometric Analysis of Apoptosis

Fractional DNA content: U87 cells (10⁵) were plated overnight at 37° C. with 5% CO₂. The cells were then treated with TNFα (10 ng/mL) and/or TMZ (100 μM). At 72 h the cells were washed in PBS and fixed in ice-cold 70% (v/v) ethanol. The cells were washed twice and incubated in RNase (1 mg/mL) for 30 m at 37° C., then incubated in propidium iodide (PI) solution (100 ug/mL) for 30 in at 4° C. Flow cytometric analysis was performed on a FACSort instrument (Becton Dickinson Immunocytometry Systems, San Jose, Calif.), and the data were analyzed using the CellQuest software (Becton Dickinson).

Annexin V binding (van Engeland, M. et al. (1998) Cytometry 31(1):1-9): At 72 h cells were washed in PBS and incubated in the dark for 15 m with binding buffer containing 5 μl of Annexin V-FITC and 5 μl of PI (Annexin V-FITC apoptosis detection kit II). The data was analyzed by Flowjo analysis software (Tree Star Inc., Ashland, Oreg.).

### Example 8

### Histological Analysis

Paraffin embedded brains were sectioned (8 μm), stained with hematoxylin and eosin and analyzed in a blinded fashion.

Terminal deoxynucleotidyl transferase-mediated dUTP-biotin nick end-labeling (TUNEL) assay was performed in accordance with the manufacturer's instructions (Chemicon) and analyzed blindly at 400× magnification by use of a computer-aided light microscope with reconstruction software (Neurolucida, Microbrightfield, Vt.). Number of TUNEL positive cells per 10⁻⁶ mm² was documented.

### Example 9

### Luciferase Assay

U87 cells (5×10³) were plated overnight and subsequently co-transfected with Ig-κB-Luc (or pE425 GL3) and the Renilla reniformis expression vector, pRL-TK, to normalize transfection efficiency, at a ratio of 10:1 using SuperFectin transfection kit (Qiagen, Valencia, Calif., USA). Twenty-four hours after transfection, the cells were pretreated with TMZ (100 μM) for 16 h, then treated with TNFα (10 ng/mL). Five hours later NF-κB (or Egr-1) and Renilla luciferase activity were measured with the Dual-Luciferase reporter assay system (Promega Corp., Madison, Wis., USA). Relative luciferase was calculated as the ratio of firefly luminescence/Renilla luminescence for each sample.

### Example 10

### Preparation of Nuclear Extracts

Confluent cultures of U87 cells were grown in complete medium and then left untreated or treated with 10 ng/mL TNFα for 20 m and 1 h+/−16 h pre-treatment with 100 μM TMZ (or 0.1% DMSO control vehicle). Cells were then washed with 10 mL ice-cold PBS, scraped from the dish, and pelleted by centrifugation at 1000 rpm for 5 m at 4° C. Cell pellets were resuspended in 400 μl of ice-cold buffer A (10 mM HEPES, pH 7.9; 10 mM KCl; 0.1 mM EDTA; 1 mM DTT; 0.5 mM phenylmethysulfonylfluoride [PMSF]; 1 μg/mL leupeptin; 5 μg/mL aprotinin) and allowed to swell on ice for 15 m. Following the addition of 25 μl of 10% NP-40, the suspension was vortexed for 10 s and centrifuged at 14,500 rpm for 1 m at 4° C. Nuclei were resuspended in 50 μl of ice-cold buffer B (20 mM HEPES, pH 7.9; 0.4 NaCl; 1 mM EDTA; 1 mM DTT; 1 mM PMSF; 25% glycerol; 1 μg/mL leupeptin; 5 μg/mL aprotinin) and incubated on ice for 15 m. The nuclear suspension was then centrifuged at 14,500 rpm for 5 m at 4° C. and the supernatant containing the nuclear proteins was transferred to a clean tube. Protein concentrations for each sample were determined by the Bradford method (Bio-Rad, Richmond, Calif., USA) and were adjusted to 2 μg/μl by the addition of buffer B.

### Example 11

### Electrophoretic Mobility Shift Assay

Assays were performed using the Promega gel shift assay system. NF-κB consensus oligonucleotide (oligo) (5′AGTTGAGGGGACTTTCCCAGGC3′) (SEQ ID NO:1) was end labeled with [γ-³²P] ATP using T4 polynucleotide kinase and incubated for 10 m at 37° C. The reaction was stopped by the addition of 1 μl of 0.5 M EDTA. Binding reactions contained the following: 5 μl nuclear extract (10 μg protein), 2 μl distilled deionized water, and 2 μl of 5× gel shift binding buffer (20% glycerol; 5 mM MgCl2; 2.5 mM EDTA; 2.5 mM DTT; 250 mM NaCl; 50 mM Tris-HCl, pH 7.5; 0.25 mg/mL poly(dI-dC)·poly(dI-dC). The reaction mixture was incubated at room temperature for 10 m, and then 1 μl (0.035 pmol) of ³²P-labeled NF-κB oligo was added. After an additional 20 m, the reaction was stopped by adding 1 μl of 10× gel loading buffer (250 mM Tris-HCl, pH 7.5; 0.2% bromophenol blue; 40% glycerol). 10 μL were loaded onto a 5% non-denaturing polyacrylamide gel and run in 0.5×TBE (45 mM Tris-HCl, 45 mM boric acid, 1 mM EDTA) for 1 h. The gel was dried under a vacuum at 80° C. for 1 h and exposed to photographic film at −70° C. For competitor reactions, 10 ng of TNFα treated U87 nuclear extract was incubated for 30 m with 50-fold excess of unlabeled NF-κB consensus sequence oligo (specific competitor) or unlabeled AP-1 consensus sequence oligo (non-specific competitor). Supershift studies were performed by 30 m pre-incubation of nuclear extracts from TNFα treated cells with antibody against p65 or p50 (Active Motif, Carlsbad, Calif., USA).

### Example 12

### Western Blot Analysis

20 μg of whole U87 cell (or nuclear) lysate was subjected to sodium dodecyl sulfate (SDS)-polyacrylamide gel electrophoresis (PAGE). Following electro-transfer, Immobilon-P membranes (Millipore Corp. Burlington, Mass., USA.) were probed with primary polyclonal antibody against IκBα, phospho-Ser32-IκBα, SAPK/JNK, phospho-Thr183/Tyr185-SAPK/JNK, p65, phospho-Ser536-p65 (Cell Signaling Technology Inc. Beverly, Md., USA) diluted 1:1000 overnight at 4° C. Anti-rabbit IgG HRP-linked secondary antibody (Cell Signaling Technology) was diluted 1:1000 in blocking buffer and applied for 1 h at room temperature. Immunoreactive bands were detected by SuperSignal enhanced chemiluminescence (ECL) (Pierce, Rockford, Ill., USA) and exposed to Kodak X-Omat film.

### Example 13

### Annexin V Binding

Cells were either un-transfected or co-transfected with pRC-CMV-p65 or pRC-CMV in the presence of a GFP plasmid at a ratio of 4:1. Under these conditions, cells expressing GFP also expressed the co-transfected plasmid (Tang, F. et al. (2002) Mol Cell Biol 22:8571-8579). Cells were then left untreated or treated as described in the figure legends. At 72 h cells were washed in PBS and incubated in the dark for 15 m with binding buffer containing 5 μl of Annexin V-FITC and 5 μl of Propidium iodide (PI). In transfected cells, annexin V binding was assessed in GFP positive cells. Data was analyzed by Flowjo analysis software (Tree Star Inc., Ashland, Oreg., USA) as described in U.S. Provisional Application No. 60/604,251 (Yamini, B. et al. (2004) Cancer Res 64:6381-6384).

### Example 14

### Protein Kinase Assay

IκK was immunoprecipitated from treated U87 cell extracts with anti-IKKβ antibody, (Santa Cruz Biotechnology, Santa Cruz, Calif., USA). The kinase activity of the immune complex was assayed at 30° C. for 30 to 60 m in 30 μl of kinase buffer (Mercurio, F. et al. (1997) Science 278:860-866) in the presence of 10 μM ATP-10 μCi [γ-³²P]ATP Dupont NEN with (GST)-IκBα (1-54) protein (purified on glutathione-agarose beads as described (DiDonato, J. A. et al. (1997) Nature 388:548-554)) as a substrate. The reaction was terminated with 4× Laemmli sample buffer and proteins resolved by SDS-12% PAGE. Kinase activity was quantified using a Phosphoimager and Equal protein loading determined by immunoblotting with anti-IκKβ antibody (Upstate USA, Charlottesville, Va., USA). The antibody-antigen complexes were visualized by the ECL detection system (Amersham, England).

### Example 15

### Measurement of Superoxide (O₂⁻) Production

U87 cells were plated at a density of 10⁶ cells in flat-bottom 6-well tissue culture plates, incubated overnight and treated as indicated in the figure legend. Cells were then washed, resuspended in PBS and 1 μL of 10 mM hydroethidine (HE) per mL cell suspension (10 μM final concentration) was added and incubated for 5 m at 37° C. Cells were harvested and analyzed on a flow cytometer (FACSort; BD Biosciences) with excitation at 488 nm and emission collected using a 620-670 nm absorbance long-pass filter. Data was analyzed by Flowjo software.

### Example 16

### Statistical Analysis

Results are expressed as mean value±SEM. Statistical significance was taken as P<0.05 using a one-tailed student t-test. Analysis of variance (ANOVA) was also employed. Kaplan-Meier survival curves were plotted for the intracranial experiment and analyzed by the Log-rank method.

### Example 17

### TMZ Induced TNFα Reduced U87MG Cell Viability

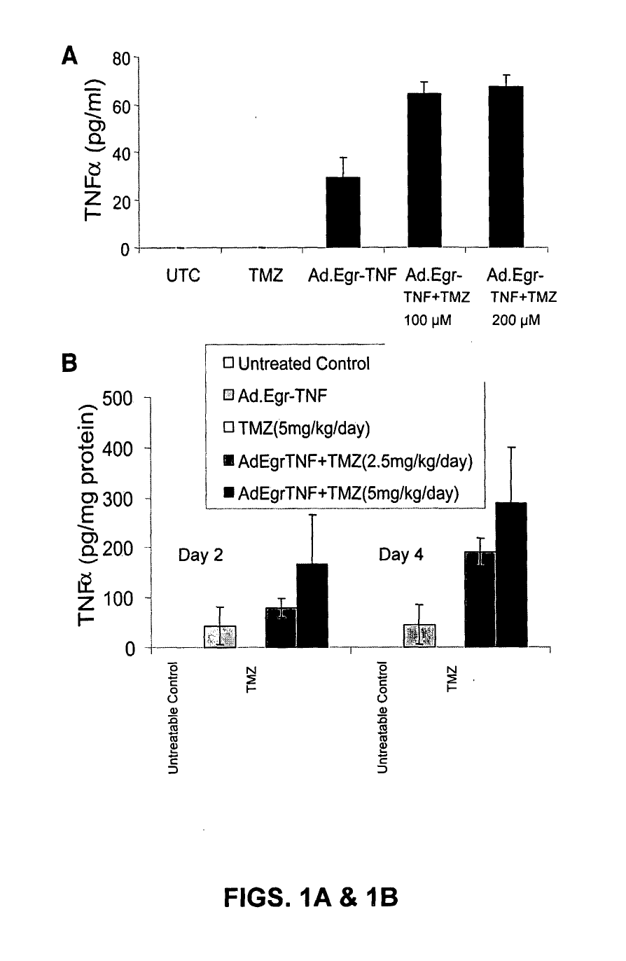

TMZ was found to induce expression of TNFα from U87 cells infected with Ad.Egr-TNF. In in vitro studies, TNFα was detected in untreated control cells or in cells treated with TMZ alone. Following Ad.Egr-TNF infection, 100 μM TMZ induced a 2.3-fold increase in TNFα expression compared to cells infected with vector alone (FIG. 1A). Hindlimb xenografts were used to evaluate in vivo induction. No TNF was detected in the untreated animals or in animals treated with TMZ alone (FIG. 1B). However, following combination treatment with Ad.Egr-TNF/TMZ, 287±111 pg TNFα/mg protein was detected at 96 h, 6.4 times more TNFα found in glioma cells of animals treated with Ad.Egr-TNF alone (n=3 animals per group, P=0.02) (FIG. 1B).

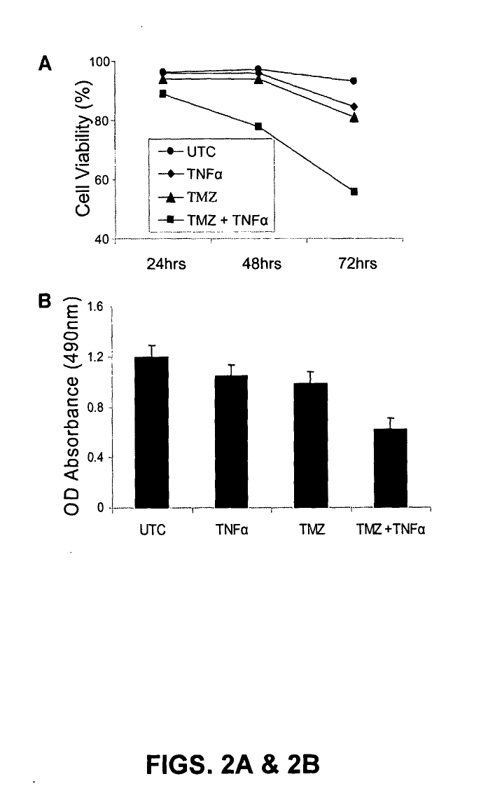

The cytotoxic effect of TNFα and TMZ on glioma cell viability was evaluated in-vitro. Minimal effects on U87 cell viability was observed in U87 cells treated with either 10 ng/mL TNFα or 100 μM TMZ alone. However, combination treatment led to a significant reduction in cell viability, the magnitude of which was greater than the sum of the reductions of either treatment alone (FIGS. 2A and B). That a synergistic interaction between TNFα, and TMZ exists is supported by analysis of variance (ANOVA) assessment (P=0.0016)

### Example 18

### Combination of TNFα and TMZ Exhibited Anti-Tumor Efficacy

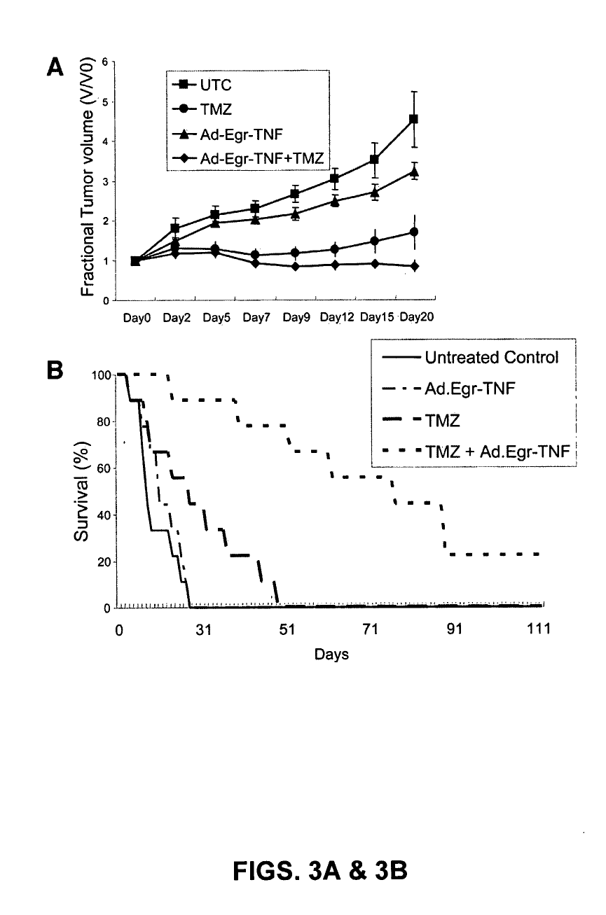

The anti-tumor efficacy of TNFα and TMZ was evaluated in hindlimb xenografts. Treatment of tumors with Ad.Egr-TNF alone did not significantly affect growth kinetics relative to growth kinetics of untreated control animals. Fractional tumor volume of animals treated with Ad.Egr-TNF and TMZ was significantly smaller compared to the fractional tumor volume of animals treated with TMZ alone (P<0.02 at day 20) (FIG. 3A). In intracranial xenograft experiments, nude mouse survival was recorded following treatment. Treatment with 20 mg/kg TMZ alone prolonged median survival over that of untreated control animals and animals treated with Ad.Egr-TNF only (28 days vs. 18 and 21 days, respectively). No mice lived past day 48. However, median survival of animals following combination treatment with Ad.Egr-TNF/TMZ was significantly increased to 76 days (P<0.001 by log-rank compared to TMZ alone) (FIG. 3B). The animals in all the treatment groups appeared healthy. Histological assessment of intracranial sections showed decreased cell density in the combined treatment group with minimal oligodendroglial toxicity and, most significantly, there was no increase in tumor necrosis when compared to either treatment alone (data not shown).

### Example 19

### TMZ and TNFα Act Synergistically to Enhance Apoptosis

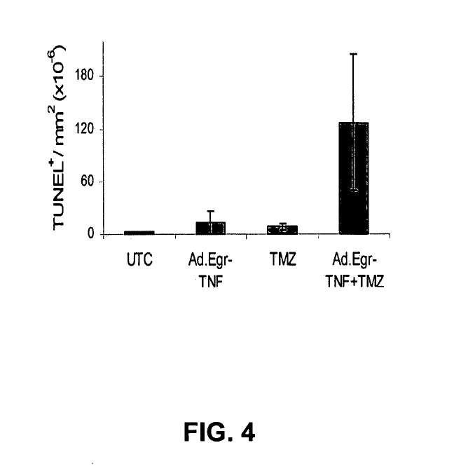

Flow cytometric (FACS) analysis of U87 cells was used to assess the fractional DNA content following treatment. As expected, TNFα alone had minimal effect on U87 cell apoptosis and TMZ alone led to an increase in the percentage of cells in G2/M phase. However, treatment with both TNFα and TMZ lead to a significant increase in the sub-G1 (hypodiploid/apoptotic) peak at 72 h, compared to either treatment alone (P<0.05). Annexin V staining of U87 cells confirmed results obtained by FACS. Combination treatment led to a 9-fold and 3.3-fold increase in annexin V positive cells compared to those treated with only TNFα or TMZ, respectively, at 72 h. The interaction between TNFα and TMZ leading to apoptosis was determined to be synergistic as assessed by ANOVA (P<0.05). To determine whether the synergistic effect of TNFα and TMZ on apoptosis observed in vitro also occurs in vivo, intracranial tumor sections, specifically, sections taken during the early stage of treatment (day 7), were evaluated using TUNEL. Tumors treated with Ad.Egr-TNF/TMZ combination had significantly more TUNEL positive cells than those treated with either TMZ or Ad.Egr-TNF alone (110±77 vs. 14±12 and 13±13 TUNEL⁺ cells/10⁻⁶ mm² respectively, P<0.05) (FIG. 4). The synergy between TNFα and TMZ provides enhanced efficacy in inhibiting growth of glioma cells over the use of TMZ alone. Although TMZ has relatively mild side effects, the maximal dose that can be safely administered is limited by hematological toxicity. The therapeutic index of TMZ can be greatly enhanced when TMZ used in a combination treatment strategy with virally delivered TNFα.

These results are unexpected in light of previous reports. Eggermont et al. reported that an observed synergistic interaction between high dose TNFα and an alkylating agent in isolated limb perfusion studies was due to increased tumor necrosis, possibly resulting from increased vascular permeability leading to an increase in intratumoral drug concentration (Eggermont, A. M. et al. (2003) Lancet Oncol 4(7):429-437). A similar pattern of tumor necrosis has also been observed when radiotherapy is combined with Ad.Egr-TNF in a flank glioma model (Staba, M. J. et al. (1998) Gene Ther 5(3):293-300). In contrast, the combination of TMZ and TNF produced no histologically detectable necrosis, and instead caused a significant increase in tumor cell apoptosis both in vitro and in vivo, whereas neither TNFα alone nor TMZ alone causes significant apoptosis in glioma cells. Considered together, these data strongly suggest that there is a direct interaction between TNFα and TMZ in glioma cells that enhances apoptosis resulting in the therapeutic benefit reported in our experiments.

A therapeutic increase in tumor cell apoptosis has been speculated to be a desirable goal of novel glioma therapies (Raza, S. M. et al. (2002) Neurosurgery 51(1):2-12; discussion 12-3) particularly because tumor necrosis has been associated with a significantly worse prognosis in GBM patients (Lacroix, M. et al. (2001) J Neurosurg 95(2):190-198). However, further studies are necessary to determine the mechanism involved in the induction of apoptosis and to evaluate whether treatment-induced apoptosis yields a greater therapeutic ratio in malignant glioma than therapeutically induced necrosis.

Mortality from malignant glioma is related primarily to recurrent disease, which is almost universally local (non-metastatic) in nature (20). For this reason, a regionally activated treatment strategy is especially suitable for treating such tumors.

### Example 20

### Temozolomide Inhibits TNFα-Induced NF-κB Transcription in Glioma Cells

Because TNFα is known to induce a pro-survival transcription factor NF-κB, the activation of which mediates resistance to other genotoxic stressors, the effect of TMZ TNFα-induced NF-κB was evaluated, as was the question of whether any such effect contributes to the apoptotic/cytotoxic interaction between TMZ and TNFα. In an NF-κB-responsive luciferase reporter assay, TMZ pre-treatment was shown to cause dose dependent inhibition of TNFα-induced NF-κB transcriptional activity in U87 glioma cells (P=0.002 TNFα+TMZ 100 μM compared to TNFα alone). TMZ has a similar effect on TNFα-induced NF-κB activity in tested human glioma cell lines T98 and U251. In contrast, TMZ activated TNFα-induced NF-κB transcriptional activity in human pancreatic and esophageal cancer cell lines (Panel, MIAPaCa-2, BxPC-3 and Seg-1). These results suggest that the inhibition of TNFα-induced NF-κB activity by TMZ may be selective for human glioma cells compared to other cancer cell types.

### Example 21

### Temozolomide Suppresses TNFα-Induced NF-κB Nuclear Translocation, Nuclear Translocation, and Activation

The results of experiments undertaken to further characterize the effect of TMZ on TNFα-induced NF-κB indicated that TMZ inhibits TNFα-induced NF-κB transcriptional activity in part by inhibiting NF-κB nuclear translocation. Additionally, NF-κB induced to translocate to the nucleus following TNFα stimulation in glioma cells was shown to contain the p65 subunit as a major component. TMZ does not directly affect TNFα-induced NF-κB DNA binding. Additionally, TMZ inhibits TNFα-induced degradation of IκBα, an upstream regulator of NF-κB. The data from the experiments performed as described above in the previous Examples showed that TNFα treatment caused complete degradation of IκBα at 15 m and that, although TMZ pretreatment had little effect on overall IκBα protein levels, it reduced TNFα-induced degradation. Furthermore, increasing concentrations of TMZ resulted in greater inhibition of TNFα-induced IκBα degradation at 15 m. These results suggest that TMZ inhibits TNFα-induced NF-κB activity at least in part by inhibiting TNFα-induced IκBα degradation. TMZ pretreatment also reduced TNFα-induced phosphorylation of IκBα, a reaction catalyzed by IOU, by 50% at 5 m. In addition, TMZ inhibits alters TNFα-induced p65 phosphorylation, which has the effect of reducing TNFα-induced NF-κB nuclear translocation. Inhibition of TNFα-induced p65 phosphorylation is overcome by overexpression of p65.

### Example 22

### Temozolomide Induces Prolonged JNK Activation that Contributes to Tumor Cell Apoptosis

Sustained JNK activation has been shown to mediate both TNFα- and DNA damage-induced apoptosis in the setting of reduced NF-κB activation (Tang, G. et al. (2001) Nature 414:313-317) (Benhar, M. et al. (2001) Mol Cell Biol 21:6913-6926). Therefore, experiments were undertaken to evaluate whether TMZ and TNFα affect JNK phosphorylation (activation) in glioma cells. As shown previously, glioma cells have baseline activation of JNK (Antonyak, M. A. et al. (2002) Oncogene 21:5038-5046). Initial experiments initially demonstrated that although TNFα transiently increased JNK activity, treatment with TMZ and TNFα led to a biphasic increase in JNK phosphorylation, with the delayed phase occurring approximately 20 h following treatment. TMZ treatment alone resulted in a progressive and delayed activation of JNK. Because JNK activation has been previously shown to occur as a result of caspase activation (Cardone, M. H. et al. (1997) Cell 90:315-323), cells were pretreated with the general caspase inhibitor, zVAD-fmk prior to assessing JNK activation. The results showed that even though zVAD completely reversed the cytotoxicity induced by combined treatment with TMZ and TNFα, it did not inhibit the delayed JNK activation induced by this combination. In fact, JNK activation was shown to be even greater following zVAD pretreatment.

Whether prolonged JNK activation is necessary for apoptosis U87 cells was assessed using annexin V binding following treatment with TNFα and TMZ in the presence of the specific JNK inhibitor SP600125. Pretreatment with SP600125 inhibited both transient and delayed JNK activation following stimulation with TMZ and TNFα, and SP600125 reverses the apoptosis induced by treatment with combination TMZ and TNFα (P<0.01 TNFα+TMZ+SP600125 compared to TNFα+TMZ). The effect of SP600125 on cell death was confirmed using an MTS assay of U87 cells at 72 h following treatment. Taken together, these data suggest that JNK activation is necessary for apoptosis following TNFα and TMZ treatment but that the INK activation, as seen with TMZ treatment alone, is not sufficient to induce apoptosis.

### Example 23

### Reactive Oxygen Species (ROS) Mediate Delayed JNK Phosphorylation and Induction of Apoptosis Following Combination TMZ and TNFα Treatment

ROS have been shown to mediate the sustained component of TNFα-induced JNK activation in cells that have a defect in NF-κB activation (Sakon, S. et al. (2003) Embo J 22:3898-3909). Whether TMZ and TNFα treatment results in accumulation of ROS in U87 cells was evaluated using the cell permeable dye hydroethidine (HE), which is oxidized by superoxide radicals (O₂^{.-}) to the fluorescent ethidium. Combination treatment with TMZ and TNFα led to a progressive increase in the accumulation of O₂^{.-} over 24 h as evidenced by an increase in the intensity of ethidium, and this increase in O₂^{.-} was significantly inhibited by pretreatment with the antioxidant NAC (P<0.05 TNFα+TMZ+NAC compared to TNFα+TMZ). Additionally, pretreatment of U87 cells with NAC reduces the delayed JNK activation induced by combination TMZ and TNFα treatment by 1.7-fold without affecting transient JNK activation.

Whether ROS play a role in apoptosis induced by combination TNFα and TMZ treatment was evaluated. Pretreatment of U87 cells with NAC had minimal effects on cell death. However, NAC significantly reversed the apoptosis induced by combination TMZ and TNFα treatment (P<0.01 TNFα+TMZ+NAC compared to TNFα+TMZ). Next, to assess a direct link between p65 and ROS, HE oxidation was evaluated in cells co-transfected with HA-p65 (or empty vector) and a GFP expression vector. The results indicate that p65 over-expression significantly reduced HE oxidation following TNFα and TMZ treatment compared to control (P<0.05).

The combination of TNFα and TMZ increase O₂^{.-} species, and inhibition of ROS results in inhibition of delayed JNK activation and apoptosis. JNK activation is downstream of ROS accumulation, which is in contrast to previous reports (Ventura, J. J. et al. (2004) Genes Dev 18:2905-2915). The results indicate that p65 inhibits the ROS accumulation induced by TNFα and TMZ treatment while having no significant effect on basal ROS production.

### Example 24

### Combination TMZ, IR and Ad.Egr-TNF Suppress Hindlimb Glioma Regrowth

IR plays a major role in the management of malignant glioma (Walker, M. D. et al. (1980) N Engl J Med 303:1323-1329) and the combined use of IR and Ad.Egr-TNF causes tumor regression by a mechanism involving both direct tumor toxicity and an indirect anti-vascular effect (Weichselbaum, R. R. et al. (2002) Lancet Oncol 3:665-671). Whether the addition of IR could significantly enhance the anti-tumor effect of TMZ and TNFα was evaluated. A complete disappearance of palpable tumor in hindlimb glioma xenografts in nude mice (10/10) treated with TMZ, IR and Ad.Egr-TNF was appreciated at 30 days following treatment 10/10 animals. In contrast, 1/10 animals treated with TMZ alone, IR alone, IR and TMZ, or TMZ/Ad.Egr-TNF groups (P<0.00001 TMZ+IR+Ad.Egr-TNF compared to Ad.Egr-TNF+TMZ). These data demonstrate a potent anti-tumor interaction in vivo.

### Example 25

### TMZ Suppresses IR- and TNFα-Induced NF-κB Activity and Nuclear Translocation In Vivo

Because IR-induced NF-κB activation has been shown to mediate radiation resistance in tumor cells, the inhibitory effect of TMZ on TNFα-induced NF-κB in vivo was evaluated. Co-treatment of glioma cells with TNFα and IR increase NF-κB transcriptional activity and nuclear translocation, and these TNFα and IR effects are inhibited by TMZ in a dose-dependent matter.

### Example 26

### Triple Therapy with Ad.Egr-TNF, TMZ and IR Leads to an Increase in Animal Survival in an Intracranial Glioma Xenograft Model

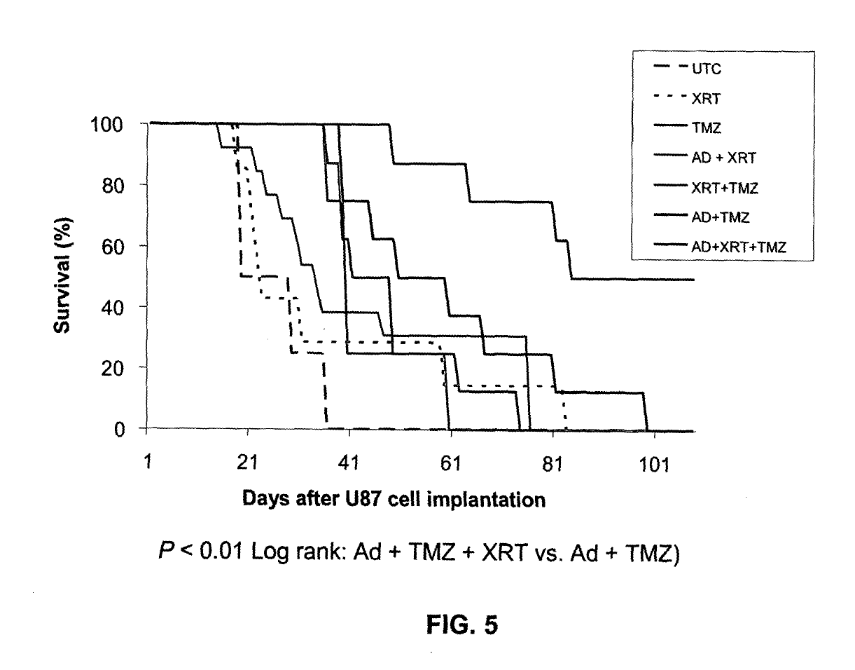

The results obtained by treating mice having a hindlimb glioma xenograft with TMZ, IR and Ad.Egr-TNF were confirmed using an intracranial glioma xenograft model. Survival of mice treated with Ad.Egr-TNF, IR and TMZ alone and in combination was evaluated. Animals treated with Ad.Egr-TNF, IR and TMZ were found to have a significant increase in median survival compared to all other treatment groups, and specifically compared to the standard anti-glioma regimen of IR and TMZ. 50% of the animals treated with TMZ, IR and Ad.Egr-TNF were still alive and appeared healthy 100 days post tumor inoculation, compared to 0% of animals in all other treatment groups (P<0.01 Ad.Egr-TNF+IR+TMZ vs. Ad.Egr-TNF+TMZ) (FIG. 5).

The use of TMZ with concomitant IR has become a standard initial strategy for the management of patients with malignant glioma. Nevertheless, prognosis for these patients remains dismal. The heterogeneous nature of malignant glioma suggests that a multimodal therapeutic strategy that incorporates conventional chemo/radiotherapy with newer experimental approaches will be needed to achieve better outcomes (Guha, A. and Mukherjee, J. (2004) Curr Opin Neurol 17:655-662). One potentially promising approach for the management of cancer has been to target death ligands, such as TNFα, to trigger apoptosis in tumor cells. This is an attractive approach as death ligands can directly activate the apoptotic cascade in part through different mechanisms than those activated by DNA damaging agents (Ashkenazi, A. (2002) Nat Rev Cancer 2:420-430). Although glioma cells have been shown to be resistant to cytotoxicity induced by the TNFα superfamily (Sakuma, S. et al. (1993) Neurooncol 15:197-208) (Knight, M. J. et al. (2004) Mol Carcinog 39:173-182), treatment in combination with chemotherapeutic agents has been shown to sensitize cells to death ligand induced cytotoxicity (Vivo, C. et al. (2003) J Biol Chem 278:25461-25467) (Yamini, B. et al. (2004) Cancer Res 64:6381-6384) (Duan, L. et al. (2001) J Neurooncol 52:23-36) (Saito, R. et al. (2004) Cancer Res 64:6858-6862).

Intracranially induced TNFα (delivered by Ad.Egr-TNF) in combination with TMZ and IR significantly increases the survival of animals bearing an intracranial glioma xenograft compared to survival of animals achieved with the current standard anti-glioma treatment regimen of IR and TMZ. Importantly, the animals treated with triple therapy appeared healthy with no early treatment-related deaths.

Activation of the transcription factor NF-κB mediates resistance to TNFα, IR and chemotherapy (Wang, C. Y. et al. (1996) Science 274:784-787) (Beg, A. A. and Baltimore, D. (1996) Science 274:782-784) (Wang, C. Y. et al. (1999) Nat Med 5:412-417). Inhibition of NF-κB activation has been shown to sensitize tumor cells to TNFα- and IR-induced apoptosis (Van Antwerp, D. J. et al. (1996) Science 274:787-789) (Yamagishi, N. et al. (1997) Int J Radiat Biol 72:157-162). Although TNFα and IR increase NF-κB activity, diverse chemotherapeutic agents have been shown to both increase and reduce NF-κB activity (Das, K. C. and White, C. W. (1997) J Biol Chem 272:14914-14920) (Campbell, K. J. et al. (2004) Mol Cell 13:853-865) (Chuang, S. E. et al. (2002) Biochem Pharmacol 63:1709-1716). This study provides the first evidence that TMZ strongly inhibits the transcriptional activity of TNFα-induced NF-κB. When used alone, TMZ slightly increases the transcriptional activity of NF-κB in glioma cells. Therefore, the observation that TNFα-induced NF-κB activity is completely inhibited by TMZ is quite unexpected. Other chemotherapeutic agents not in general clinical use were previously reported to inhibit TNFα-induced NF-κB activity (Ichikawa, H. et al. (2005) J Immunol 174:7383-7392). Much emphasis and research is currently focused on the development of clinically useful inhibitors of the NF-κB activation pathway (Karin, M. et al. (2004) Nat Rev Drug Discov 3:17-26) (Aggarwal, B. B. (2004) Cancer Cell 6:203-208). TMZ is a commonly used DNA alkylator with a favorable toxicity profile and its inhibition of TNFα- and IR-induced NF-κB potentially represents a novel and clinically useful mechanism by which death ligands and conventional DNA damaging agents can be combined in the management of malignant glioma. The results disclosed herein indicate that TMZ suppresses TNFα-induced NF-κB activity in several glioma cell lines (U251, T98 and U87 cells), but not in pancreatic or esophageal cancer cell lines, which suggests that TMZ-mediated inhibition of NF-κB activation may be specific for glioma cells.

All patents, publications and references cited herein are hereby fully incorporated by reference. In case of conflict between the present disclosure and incorporated patents, publications and references, the present disclosure should control.

We claim:

1. A method of synergistically inhibiting growth of a glioma cell comprising contacting the cell with a growth inhibiting amount of temozolomide and TNFα.

2. The method of claim 1, wherein the TNFα is provided by a vehicle comprising or expressing TNFα.

3. The method of claim 2, wherein the vehicle comprises an engineered adenovirus comprising a nucleotide sequence encoding TNFα operably linked to a chemoinducible or radioinducible promoter.

4. The method of claim 3, wherein the promoter comprises the CArG elements of the Egr-1 promoter.

5. The method of claim 4, wherein the temozolomide induces expression of TNFα.

6. The method of claim 1, wherein TNFα or temozolomide are administered to a human cancer patient in amounts effective to inhibit the growth of the glioma cells.

7. The method of claim 1, wherein contacting the cell with TNFα and temozolomide increases cytotoxicity or apoptosis, relative to that of glioma cells treated with only TNFα or only temozolomide.

8. The method of claim 1, wherein the temozolomide inhibits TNFα-induced transcription, nuclear translocation, or activation of NFκB.

9. The method of claim 1, wherein temozolomide increases c-Jun N-terminal kinase activity.

10. The method of claim 1, wherein temozolomide inhibits p65 phosphorylation.

11. The method of claim 1, further comprising irradiating the cell.

12. A method of synergistically inhibiting growth of a glioma in a human cancer patient comprising administering to the patient temozolomide and a vehicle comprising or expressing TNFα, wherein the vehicle is administered directly to the glioma.

13. The method of claim 12, wherein the vehicle comprises an engineered adenovirus comprising a nucleotide sequence encoding TNFα operably linked to a chemoinducible or radioinducible promoter.

14. The method of claim 12, wherein the vehicle genetic construct is administered intratumorally.

15. The method of claim 13, wherein the promoter comprises the CArG elements of the Egr-1 promoter.

16. The method of claim 12, wherein glioma volume is reduced following administration of temozolomide and the vehicle comprising or expressing TNFα.

17. The method of claim 12, wherein administration of temozolomide and a vehicle comprising or expressing TNFα is correlated with increased glioma cell cytotoxicity or apoptosis, relative to that of glioma cells treated with only TNFα or only temozolomide.

18. The method of claim 12, wherein the temozolomide induces expression of TNFα.

19. The method of claim 12, wherein the administration of the temozolomide and a vehicle comprising or expressing TNFα is not correlated with increased necrosis.

20. The method of claim 12, wherein the glioma is physically associated with the brain, spinal cord, or optic nerve.

21. The method of claim 12, wherein the survival of the patient exceeds that of a control or control population receiving temozolomide alone.

22. The method of claim 12, further comprising irradiating the glioma with ionizing radiation.

23. The method of claim 12, wherein the glioma cell is a malignant glioma cell.

24. A method of synergistically inhibiting growth of a glioma in a human cancer patient comprising administering to the patient temozolomide, a vehicle comprising or expressing TNFα, and radiation, wherein the vehicle and radiation are administered directly to the glioma.

25. The method of claim 24, wherein the vehicle comprises an engineered adenovirus comprising a nucleotide sequence encoding TNFα operably linked to a chemoinducible or radioinducible promoter.

26. The method of claim 24, wherein the vehicle genetic construct is administered intratumorally.

27. The method of claim 25, wherein the promoter comprises the CArG elements of the Egr-1 promoter.

28. The method of claim 24, wherein glioma volume is reduced following administration of temozolomide, the vehicle comprising or expressing TNFα, and radiation.

29. The method of claim 24, wherein the survival of the patient exceeds that of a control or control population receiving a subcombination of temozolomide, a vehicle comprising or expressing TNFα, and radiation.

30. A pharmaceutical combination comprising temozolomide and a vehicle comprising or expressing TNFα.

31. The combination of claim 30, further comprising a radionuclide.

32. A therapeutic combination comprising temozolomide, a vehicle comprising or expressing TNFα, and radiation.
